(19) United States
(12) Patent Application Publication
(10) Pub. No.: US 2004/0037844 A1
(43) Pub. Date: Feb. 26, 2004

(54) Vaccine

(76) Inventors: Graham A.W Rook, London (GB); Geok T. Seah, Ocean Park (SG)

Correspondence Address: NIXON & VANDERHYE, PC, 1100 N GLEBE ROAD, 8TH FLOOR, ARLINGTON, VA, 22201-4714, US

(21) Appl. No.: 10/399,565

(22) PCT Filed: Oct. 15, 2001
(86) PCT No.: PCT/GB01/04572

(30) Foreign Application Priority Data
Oct. 19, 2000 (GB) 0025694.1

(51) Int. Cl. A61K 39/00; A61K 38/20

(52) U.S. Cl. 424/185.1; 424/85.2

(57) ABSTRACT

The present invention relates to vaccine compositions comprising a Th2 inducing antigen, and optionally an adjuvant, wherein said optional adjuvant induces T-helper cell 1 (Th1). The invention further relates to methods for selecting antigens for use in vaccine compositions.

### FIELD OF THE INVENTION

The present invention relates to vaccines against mycobacterial disease, and to the identification of antigen(s) useful in such vaccines.

### BACKGROUND TO THE INVENTION

Immunity to tuberculosis is mediated by T-helper 1 (Th1) lymphocytes which recognise antigens from M. tuberculosis and secrete cytokines including interleukin 2 (IL-2) and Interferon gamma (IFN-γ). These cells activate macrophages and enhance formation of cytotoxic T cells which are the effector systems that lead to killing of the mycobacteria (Orme et al., 1993; Orme et al., 1993; Silver et al., 1998; Stenger et al., 1998).

Prior art attempts to identify antigen(s) from M. tuberculosis that are suitable for use in vaccines rely on the selection criterion of the antigen(s) being able to evoke strong Th1 responses.

According to the prior art, lymphocytes from patients, immune individuals or immunised animals are cultured with fractionated or cloned antigens, and evidence of activation of Th1 cells is sought as an indication of their suitability for use as antigen(s) in a vaccine.

This evidence is usually sought by looking for production of IFNγ (for example (Lindblad et al., 1997))

There is clearly a need to identify antigens suitable for use in vaccines against M. tuberculosis. The prior art focusses on the the ability to evoke a Th1 response as an indicator of suitablility for use in vaccines.

### SUMMARY OF THE INVENTION

The present invention is based on the surprising finding that progressive tuberculosis is attributable to the ability of M. tuberculosis to induce not only the dominant Th1 response, but also a small response mediated by Th2 lymphocytes (including secretion of IL-4, IL-13) which, when superimposed upon the Th1 response, causes immunopathology, and impairs the bactericidal functions of the Th1 effector mechanisms.

The methods and vaccines of the present invention utilise this surprising finding. It enables the selection of antigens for incorporation into a vaccine by identifying those that tend to evoke a Th2 response. These are the antigens which are causing the immune response to malfunction.

According to the present invention, these antigens are identified and used in vaccine(s) in such a way as to pre-empt inducion of a Th2 response when a subject subsequently meets them in/on M. tuberculosis.

Thus, the present invention relates to methods for identifying Th2-inducing antigens. The present invention further relates to vaccine formulations containing these Th2-inducing antigens.

### DETAILED ASPECTS OF THE PRESENT INVENTION

In one aspect, the present invention relates to a vaccine composition comprising a T-helper cell 2 (Th2) inducing antigen.

In another aspect, the present invention relates to a vaccine composition comprising a Th2 inducing antigen, and an adjuvant, wherein said adjuvant induces T-helper cell 1 (Th1).

In another aspect, the present invention relates to a vaccine as described herein, wherein said adjuvant comprises IL-12.

In another aspect, the present invention relates to a vaccine as described herein, wherein said adjuvant comprises Mycobacterium vaccae, or a part thereof.

In another aspect, the present invention relates to the use of an adjuvant in a vaccine, wherein said adjuvant induces T-helper cell 1 (Th1).

In another aspect, the present invention relates to the use of an adjuvant in a vaccine, wherein said adjuvant comprises IL-12.

In another aspect, the present invention relates to the use of an adjuvant in a vaccine, wherein said adjuvant comprises Mycobacterium vaccae, or a part thereof.

In another aspect, the present invention relates to a method of identifying an antigen for use in a vaccine for mycobacterial disease, said method comprising providing a candidate antigen, providing a first and a second sample of peripheral blood mononuclear cells (PBMCs), contacting said antigen with said first sample of PBMCs, monitoring the expression levels of IL-4 in said first and second samples of PBMCs, and comparing said expression levels of IL-4 in the two samples, wherein antigens inducing raised expression of IL-4 in the first sample of PBMCs as compared to the levels of expression of IL-4 in the second (untreated) sample of PBMCs are identified as useful in said vaccine.

In another aspect, the present invention relates to a method of identifying an antigen for use in a vaccine for mycobacterial disease, said method comprising providing a candidate antigen, providing a first and a second sample of peripheral blood mononuclear cells (PBMCs), contacting said antigen with said first sample of PBMCs, monitoring the expression of CD30 in said first and second samples of PBMCs, and comparing said expression levels of CD30 in the two samples, wherein antigens inducing raised expression of CD30 in the first sample of PBMCs as compared to the levels of expression of CD30 in the second (untreated) sample of PBMCs are identified as useful in said vaccine.

In another aspect, the present invention relates to a method of identifying an antigen for use in a vaccine for mycobacterial disease as described herein, wherein the mycobacterial agent comprises M. tuberculosis.

In another aspect, the present invention relates to an antigen identified using a method as described herein.

In another aspect, the present invention relates to a vaccine comprising an antigen identified as described herein.

In another aspect, the present invention relates to a pharmaceutical composition comprising an antigen as described herein, and optionally a pharmaceutically acceptable carrier, diluent or excipient.

In another aspect, the present invention relates to a method for immunising a subject comprising administering a vaccine as described herein.

For ease of reference, these and further aspects of the present invention are now discussed under appropriate section headings. However, the teachings under each section are not necessarily limited to each particular section.

Preferable Aspects

Preferably, the adjuvant(s) used in the vaccines of the present invention is capable of inducing Th1. Preferably, said adjuvant may be IL-12.

Preferably, the antigen(s) used in the vaccines of the present invention are capable of inducing a Th2 response.

Preferably, the antigens used in the vaccines of the present invention are capable of inducing IL-4 mRNA and/or IL-4 dependent CD30 expression in normal human mononuclear cells in vitro.

Preferably, the invention relates to vaccine(s) that contain Th2-inducing antigen(s) in Th1-inducing formulation(s).

Advantages

It is an advantage of the present invention that immunological responses are evoked against Th2-inducing antigens.

It is an advantage of the present invention that antigens may be selected for use in vaccines according to their ability to induce a Th2 response.

Tuberculosis

Tuberculosis is caused by a mycobacterial agent. The term ‘mycobacterial agent’ as used herein includes the M. tuberculosis bacterium.

In patients with progressive tuberculosis there is not only a Th1 lymphocyte response, but as dislcosed herein, there is clear evidence of an inappropriate Th2 response, involving lymphocytes that secrete type 2 cytokines, including interleukin 4 (IL-4) and interleukin 13 (IL-13).

The prior art indicates that this Th2 component is controversial, and its existence is disputed. A further prior art study found that M. tuberculosis caused increased expression of CD30 on human lymphocytes in vitro, but this document (Munk et al., 1997) taught that the increased CD30 expression was attributed to an IL-4-independent pathway.

However, it is surprisingly disclosed herein that the increase in Th2 cytokine expression is significant. Furthermore, our disclosure that the CD30 expression is indeed IL-4-driven is unexpected.

Immunological Response to Mycobacterial Agents

Both IL-4 and IL-13 mRNAs are expressed at significantly higher levels in fresh unstimulated peripheral blood mononuclear cells from tuberculosis patients (1.4 and 1.2 logs higher respectively) than in cells from matched tuberculin-positive controls. The biological significance of this observation is indicated by significant correlations with radiologic extent of disease, and with a marker of increased type-2 cytokine activity in vivo—serum IgE (Seah et al., 2000). Previous uncertainty is attributable to methodological difficulties in the prior art, and lack of awareness of an IL-4 splice variant, IL-4δ2. This observation has been confirmed using a flow cytometric technique.

Furthermore, it is shown that there are components of M. tuberculosis that will drive a Th2 component in the in vitro lymphocyte response of peripheral blood mononuclear cells from normal healthy donors. These components are either not present in a control organism, M. vaccae, or present at much lower levels. Clearly, such components are examples of antigens useful in the present invention.

This Th2 component of the response to M. tuberculosis can be measured in vitro in numerous ways, for example by assaying the increase in expression of CD30 on lymphocytes, using flow cytometry, in the presence and in the absence of a neutralising antibody to IL-4. This facilitates the induction of expression of CD30 due to IL-4 to be assessed, without factors other than IL-4 which may contribute to the expression of CD30 adversely affecting the assay.

Another example of a way in which the Th2 component of the response to M. tuberculosis can be measured in vitro in is by assaying expression of IL-4. This can be accomplished for example by extracting RNA from the cultured cells, and performing a quantitative nested reverse transcription polymerase chain reaction (RT-PCR) for mRNA encoding IL-4. This is preferably done with primers such as those described in Seah and Rook (Seah & Rook, 1999), so that only mRNA encoding IL-4 is measured, and not that encoding IL-4δ2.

These techniques are discussed more fully below, such as in the Examples section.

Biological Role of the Th2 Component

Without wishing to be bound by theory, it is believed that the following discussion assists in the illustration of the present invention.

If experimental animals are preimmunised with mycobacterial antigens in such a way that there is a Th2 response present, before challenge with virulent M. tuberculosis, there is accelerated disease and more rapid death than in entirely unimmunised animals (Hernandez-Pando et al., 1997; Lindblad et al., 1997). This may be least partly due to increased toxicity of tumour necrosis factor alpha (TNFα) in mixed Th1/Th2 sites of mycobacterium-induced inflammation (Hernandez-Pando & Rook, 1994).

A similar increase in immunopathology and fibrosis is seen in other models of infection when there is a simultaneous Th2 component. Examples of such are schistosomiasis (Wynn et al., 1995) and Triczinella spiralis (Lawrence et al., 1998).

This is in good agreement with the observation that the extent of Th2 activation in human tuberculosis is directly related to the tissue damage and cavitation (Seah et al., 2000; van Crevel et al., 2000). As well as promoting immunopathology, the increased production of Th2 cytokines such as IL-4 and IL-10 causes decreased macrophage function and impairs bactericidal activity (Powrie et al., 1993).

Thus, when a non-vaccinated subject encounters M. tuberculosis, the lymphocyte response is dominated by Th1 cells, but the Th2-inducing components simultaneously induce activation of some Th2 cells. If these become too numerous or active, the Th1 response cannot operate correctly as protective immunity. Therefore, vaccines according to the present invention advantageously inhibit these Th2-inducing components from inducing Th2, and preferably divert, bias or skew the reponse to them into Th1 mode.

T Helper Cells

The term ‘Th1’ as used herein refers to a type 1 T-helper cell (Th1). The term may also be used herein to refer to the response mediated by or through such a cell type. Such a response may include one or more of the secretion of Interleukin-2 (1L-2), the secretion of Interferon-gamma (IFN-γ), activation of macrophage, activation of cytotoxic T-cells, or any other Th1-associated event. Thus, the term ‘Th1 ’ may include Th1 cell(s) as well as the immune response(s) which such cell(s) produce.

The term ‘Th2’ as used herein refers to a type 2 T-helper cell (Th2). The term may also be used herein to refer to the response mediated by or through such a cell type. Such a response may include one or more of the secretion of Interleukin-4 (IL-4), the secretion of the splice variant interleukin IL-4δ2, the secretion of Interleukin-13 (IL-13), increase in levels of cell determinant 30 (CD30) on lymphocytes, increase in levels of Immunoglobulin-E (IgE) in the blood, or any other Th2-associated event. Thus, the term ‘Th2’ may include Th2 cell(s) as well as the immune response(s) which such cell(s) produce.

Vaccines

The preparation of vaccines which contain one or more substances as an active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the active ingredient(s) encapsulated in liposomes. The active ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine may contain minor amounts of auxiliary substances such as wetting or emulsifying agents and pH buffering agents.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

The term “administered” includes delivery by delivery mechanisms including injection, lipid mediated transfection, liposomes, immunoliposomes, lipofectin, cationic facial amphiphiles (CFAs) and combinations thereof, or even viral delivery. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

The term “co-administered” means that the site and time of administration of each of the antigen and/or antigenic determinants of the present invention and an additional entity such as an adjuvant(s) are such that the necessary modulation of the immune system is achieved. Thus, whilst the antigen and adjuvant may be administered at the same moment in time and at the same site, there may be advantages in administering the antigen and/or antigenic determinants at a different time and to a different site from the adjuvant. The antigen and/or antigenic determinants and adjuvant may even be delivered in the same delivery vehicle—and the antigen and/or antigenic determinants and adjuvant(s) may be coupled and/or uncoupled and/or genetically coupled and/or uncoupled.

The antigen, antigenic determinant, peptide or homologue or mimetic thereof may be administered separately or co-administered to the host subject as a single dose or in multiple doses.

The vaccine composition of the invention may be administered by a number of different routes such as injection (which includes parenteral, subcutaneous and intramuscular injection) intranasal, mucosal, oral, intra-vaginal, urethral or ocular administration. Preferably administration is by injection. For vaccination the vaccine composition can be provided in 0.1 to 0.2 ml of aqueous solution, preferably physiological saline, and administered parenterally, for example by intradermal inoculation. The vaccine according to the invention is preferably injected intracutaneously. Slight swelling and redness, sometimes also itching may be found at the injection site. The mode of administration, the dose and the number of administrations can be optimised by those skilled in the art in a known manner.

Antigens

As used herein, an “antigen” means an entity which, when introduced into an immunocompetent host, stimulates the production of a specific antibody or antibodies that can combine with the entity, and/or stimulates the relevant Th response, such as Th2. The antigen may be a pure substance, a mixture of substances or soluble or particulate material (including cells or cell fragments or cell sonicate). In this sense, the term includes any suitable antigenic determinant, cross reacting antigen, alloantigen, xenoantigen, tolerogen, allergen, hapten, and immunogen, or parts thereof, as well as any combination thereof, and these terms are used interchangeably throughout the text.

The term “antigenic determinant” as used herein refers to a site on an antigen which is recognised by an antibody or T-cell receptor, or is responsible for evoking the Th2 response. Preferably it is a short peptide derived from or as part of a protein antigen. However the term is also intended to include glycopeptides and carbohydrate epitopes. The term also includes modified sequences of amino acids or carbohydrates which stimulate responses which recognise the whole organism.

It is advantageous if the antigenic determinant is an antigenic determinant of the infectious agent (such as a mycobacterium) which causes the infectious disease.

The present invention provides method(s) for identifying antigen(s) for use in protective and therapeutic vaccines against mycobacterial disease, particularly tuberculosis.

The term “identify” as used in relation to the identification of antigen(s) for use in vaccines according to the present invention, (or for use in the production of such vaccines), is understood to include selecting, validating, confirming, assaying, testing, assessing or otherwise investigating candidate antigens and thereby determining their suitability for use in vaccine(s) according to the present invention. These techniques are further discussed herein.

A “protective” or “prophylactic” vaccine is a vaccine which is administered to naive individuals to prevent disease development, such as by stimulating active immunity.

A “therapeutic” vaccine is a vaccine which is administered to individuals with an existing infection to reduce or minimise the infection or to abrogate the immunopathological consequences of the disease.

A suitable antigen according to the present invention preferably induces Th2 response(s).

A suitable antigen according to the present invention preferably has one or more of the following properties;

capable of inducing IL-4 (interleukin-4)

capable of inducing CD30 (cell determinant 30)

capable of inducing IL-4δ2

capable of inducing IL-13

capable of inducing IL-5

capable of inducing IL-10

capable of inducing IL-6

capable of inducing IgE (immunoglobulin E)

capable of inducing IgG4 (immunoglobulin gamma 4)

capable of inducing other Th-2 associated response(s).

More preferably an antigen according to the present invention has one or more of the following properties;

capable of inducing IL-4 (interleukin-4)

capable of inducing CD30 (cell determinant 30)

capable of inducing IL-4δ2

capable of inducing IL-13

capable of inducing IL-5

capable of inducing IgE (immunoglobulin E)

capable of inducing IgG4 (immunoglobulin gamma 4)

capable of inducing other Th-2 associated response(s).

More preferably an antigen according to the present invention has one or more of the following properties;

capable of inducing IL-4 (interleukin-4)

capable of inducing CD30 (cell determinant 30)

capable of inducing IgE (immunoglobulin E)

capable of inducing IgG4 (immunoglobulin gamma 4)

capable of inducing other Th-2 associated response(s).

More preferably an antigen according to the present invention has one or more of the following properties;

capable of inducing IL-4 (interleukin-4)

capable of inducing CD30 (cell determinant 30)

capable of inducing other Th-2 associated response(s).

These properties may be tested according to methods discussed herein, for example by monitoring the effects of candidate antigen(s) on normal human mononuclear cells in vitro.

Thus, the suitability of a candidate antigen may be assessed by measurement of IL-4 mRNA levels in response to contacting normal human mononuclear cells with candidate antigen(s) in vitro. Candidate antigens inducing increased IL-4 mRNA levels are suitable for use in vaccines according to the invention.

The suitability of a candidate antigen may be assessed by measurement of CD30 expression in response to contacting normal human mononuclear cells with candidate antigen(s) in vitro. Candidate antigens inducing increased CD30 levels are suitable for use in vaccines according to the invention.

The suitability of a candidate antigen may be assessed by measurement of IL-4 dependent CD30 expression in response to contacting normal human mononuclear cells with candidate antigen(s) in vitro. This may be accomplished for example by subtracting CD28 mediated CD30 expression produced via CD80, CD86 by using CTLA-4/Fc protein, which inhibits CD28/CD80/CD86 induced expression of CD30, and allows an assessment of the increase in CD30 expression which is attributable to or dependent on IL-4 signalling. As an alternative to using CTLA-4/Fc, neutralising anti-IL-4 antibody may be used to determine the IL-4 induced CD30 expression. Candidate antigens inducing increased IL-4 dependent CD30 levels are suitable for use in vaccines according to the invention.

The suitability of a candidate antigen may be assessed by measurement of IL-10 production in response to contacting normal human mononuclear cells with candidate antigen(s) in vitro. Candidate antigens inducing increased IL-10 levels are suitable for use in vaccines according to the invention.

The suitability of a candidate antigen may be assessed by measurement of IL-13 production in response to contacting normal human mononuclear cells with candidate antigen(s) in vitro. Candidate antigens inducing increased IL-13 levels are suitable for use in vaccines according to the invention.

The suitability of a candidate antigen may be assessed by measurement of IL-4δ2 production in response to contacting normal human mononuclear cells with candidate antigen(s) in vitro. Candidate antigens inducing increased IL-4δ2 levels are suitable for use in vaccines according to the invention.

The suitability of a candidate antigen may be assessed by measurement of IgE production in response to introduction of candidate antigen(s) into an immune system such as by inoculation of a test subject such as a mammalian test subject such as a mouse. Candidate antigens inducing increased IgE levels are suitable for use in vaccines according to the invention.

The suitability of a candidate antigen may be assessed by measurement of levels of any other suitable Th2 marker in response to candidate antigen(s). Candidate antigens inducing increased Th2 response(s) are suitable for use in vaccines according to the invention.

Preferred antigens according to the present invention are those that induce IL-4 mRNA and/or IL-4 dependent CD30 expression in normal human mononuclear cells in vitro.

M. tuberculosis sonicate (MtbS) serves as an example of an antigen according to the present invention.

Adjuvants

The term ‘adjuvant’ has its normal meaning as used herein, ie. an entity capable of augmenting or participating in the influencing of an immune response. An adjuvant is any substance or mixture of substances that assists, increases, modifies or diversifies the immune response to an antigen. The adjuvant substances may include polypeptides as discussed herein, for example an adjuvant of the present invention may be a polypeptide based molecule or mimetic thereof which itself stimulates an immune response. This is discussed in more detail below. Preferred adjuvants are IL-12, and/or M. vaccae and/or Th1 inducing entities.

The vaccine compositions of the present invention may comprise one or a combination of adjuvants which enhance the effectiveness of the vaccine. Examples of additional adjuvants which, may be effective include but are not limited to: aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium pairvm (Propioniobacterium acnes), Bordetella pertussis, Mycobacterium vaccae, polyribonucleotides, sodium alginate, lanolin, lysolecithin, vitamin A, interleukins such as interleukin-12, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Only aluminum hydroxide is approved for human use.

In a preferred aspect, the present invention relates to antigen(s) mixed with a Th1-inducing adjuvant. Examples of such adjuvants include IL-12, M. vaccae, and are discussed herein.

In a preferred aspect, the present invention relates to vaccine that contains one or more Th2-inducing antigen(s) in a Th1-inducing formulation.

Th1-inducing adjuvant could be IL-12, or Mycobacterium vaccae or part(s) thereof such as sonicate, cell extract, whole cells or analogous material. ‘Th1-inducing adjuvant’ may comprise one or more other adjuvant(s) with Th1-inducing properties.

Thus, the present invention relates to a Th1-inducing adjuvant.

In a preferred embodiment, the present invention relates to a vaccine that contains Th2-inducing antigens in a Th1-inducing adjuvant formulation.

Examples of such a vaccine composition include a Th2 inducing antigen mixed with a Th1-inducing adjuvant which adjuvant could be IL-12, or Mycobacterium vaccae or other adjuvant with Th1-inducing properties.

Pharmaceutical Compositions

The present invention also provides a pharmaceutical composition comprising a therapeutically effective amount of the agent of the present invention (such as vaccine and/or adjuvant composition(s) as discussed herein) and a pharmaceutically acceptable carrier, diluent or excipients (including combinations thereof).

The pharmaceutical composition may comprise two components—wherein a first component comprises antigen and a second component which comprises adjuvant thereof. The first and second component may be delivered sequentially, simultaneously or together, and even by different administration routes.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes. Preferably the formulation is of injectable form.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Pharmaceutical Combinations

The agent of the present invention may be administered with one or more other pharmaceutically active substances. By way of example, the present invention covers the simultaneous, or sequential treatments with an agent according to the present invention and one or more steroids, analgesics, antivirals or other pharmaceutically active substance(s).

It will be understood that these regimes include the administration of the substances sequentially, simultaneously or together.

The present invention will now be described, by way of example only, in which reference will be made to the following figures:

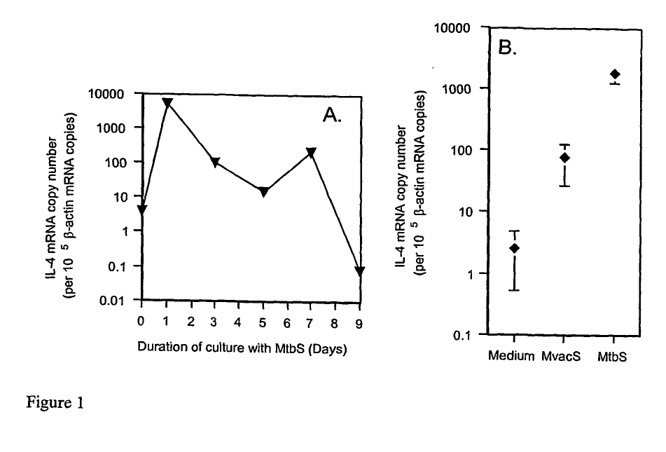

FIG. 1, which shows graphs,

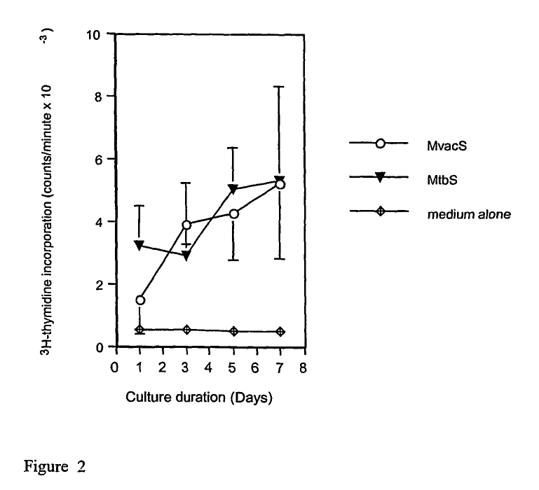

FIG. 2 which shows graphs,

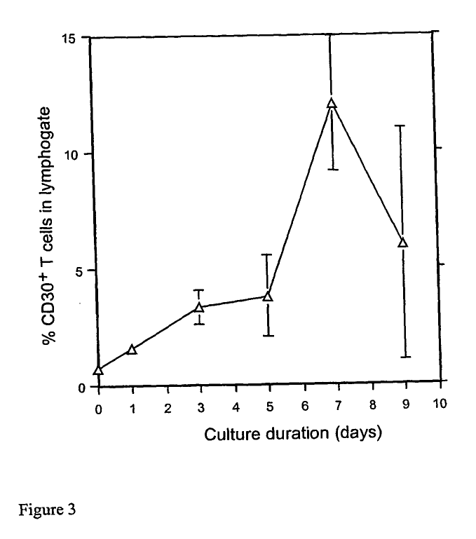

FIG. 3 which shows a graph,

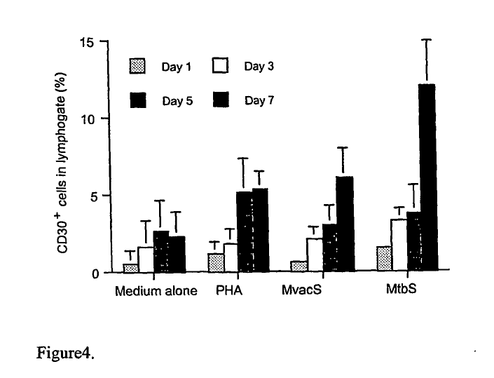

FIG. 4 which shows graphs,

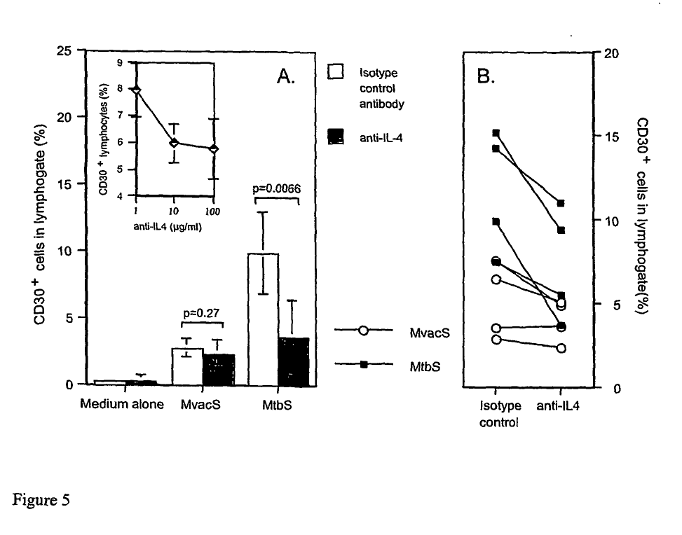

FIG. 5 which shows graphs, and

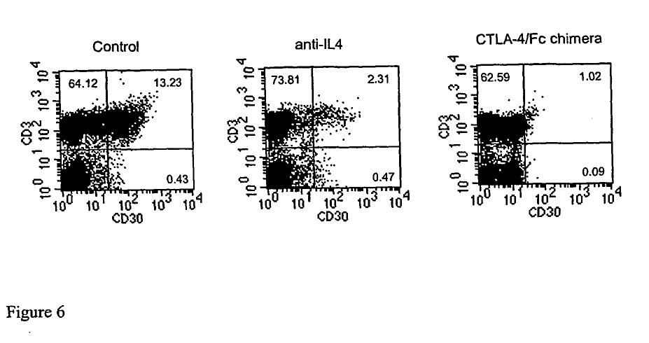

FIG. 6, which shows dot plots.

These figures are described in more detail below in the following Example sections.

### EXAMPLES

1) M. tuberculosis Sonicate Drives Increased Expression of IL-4 mRNA in Normal Human Peripheral Blood Mononuclear Cells In Vitro. (FIG. 1)

Peripheral blood mononuclear cells were cultured in vitro with ultrasonically disrupted M. vaccae (MvacS 50 μg/ml) or M. tuberculosis, (MtbS 50 μg/ml). Control cells were incubated in medium only. On the indicated days replicate wells were harvested and the mRNA was extracted. The copy number of mRNA encoding IL-4 was assayed as described in detail (Seah & Rook, 1999).

Results

M. tuberculosis induced significantly more IL-4 mRNA than did M. vaccae and this was biphasic, with peaks at 24 hrs and 7 days.

FIG. 1. IL-4 mRNA expression in response to mycobacterial sonicates.

A. IL-4 mRNA expression (by RT-PCR) in NAC cultured for varying time points with M. tuberculosis sonicate (50 μg/ml). This figure represents mean results of duplicate experiments performed with cells of one donor (<5% difference between duplicate results). Since the highest IL-4 mRNA levels were measured at 24 h, this time point was used for the next experiment (shown in B) to compare results from cells of three other donors.

B. IL-4 mRNA expression in NAC harvested after 24 h culture with culture medium alone or with either of the mycobacterial sonicates. MtbS-stimulated cells expressed significantly higher levels of IL-4 mRNA than unstimulated (p=0.0004) or MvacS-stimulated cells (p=0.0005). Statistics by t-test for independent samples. Duplicate experiments were performed using cells for each donor and the results represent the means and 2 SD of data from three different donors. Where error bars are not shown, the error values fall within the symbols.

2) The Greater Induction of IL-4 mRNA by M. tuberculosis is Not Secondary to a Greater Overall Proliferative Effect (FIG. 2).

The lymphoproliferative response evoked in normal human peripheral blood mononuclear cells by MvacS (50 μg/ml), MtbS (50 μg/ml) or culture medium alone, was assessed by H3-thymidine incorporation.

Result

The two mycobacterial sonicate preparations did not differ in their overall stimulation of the cells. Therefore the greater induction of IL-4 mRNA by the MtbS is qualitative difference in its immunological properties.

FIG. 2. Lymphocyte proliferation in response to MvacS (50 μg/ml), MtbS (50 μg/ml) or culture medium alone. The difference in proliferative responses to the two mycobacterial sonicates was not significant at any time point (p>0.1 by t-test for independent samples). The results represent means and 2 SD of data from triplicate wells in one experiment which is representative of three separate experiments showing similar results, performed with cells from different donors. Where error bars are not shown, the error values fall within the symbols.

3) M. tuberculosis Sonicate Induces Expression of CD30 on Normal Peripheral Blood Mononuclear Cells; Time Course (FIG. 3)

The expression of CD30 on lymphocytes cultured in the presence of MtbS was studied at intervals over a period of nine days. Experiments consistently showed that almost all (>98%) of the CD30+ cells in the lymphogate were T cells (CD3+). CD30 expression peaked at 7 days.

FIG. 3. Kinetics of CD30 expression in MtbS-stimulated cultures. NAC were harvested at various time-points for immunostaining and lymphogated cells were analysed by flow cytometry. Mean results and 2 SD of triplicate experiments performed using cells from one donor are shown, and are representative of three separate experiments performed using cells from different donors. Where error bars are not seen, the error values fall within the symbols.

4) M. tuberculosis Sonicate-Induced Expression of CD30 on Normal Peripheral Blood Mononuclear Cells; Comparison of M. tuberculosis With Other Stimuli (FIG. 4)

The effect of MtbS in inducing CD30 expression was compared with that of MvacS and phytohaemagglutinin (PHA). The effect of PHA was tested, because it was of interest to know whether the CD30 expression was MtbS-specific or merely the result of non-specific T cell activation. PHA-induced CD30 expression in lymphocyte-gated cells rose earlier than that in MtbS-treated cells but peaked at 5% on Day 5, reaching a plateau thereafter. MvacS-treated cells reached similar levels on Day 7, but MtbS-treated cells expressed significantly higher levels of CD30 at the peak on Day 7 (FIG. 4), with a mean of 12% in four donors, as compared to 5.1% in MvacS-treated cells.

FIG. 4. CD30 expression in response to different stimulation conditions. PBMCs were cultured in the presence of media alone, phytohaemagglutinin (PHA), MvacS or MtbS as previously described, and CD30 expression on NAC determined at various time-points. Although PHA induced CD30 expression earlier, the highest levels of CD30 expression during the period of observation were induced by MtbS (p<0.002 on Day 7 in comparison to all other antigens). The figure shows means and 2 SD of triplicate assays from one representative experiment out of two performed using cells from different donors.

5) Effect of Neutralising Antibody to IL-4 on Induction of CD30 Expression by M. tuberculosis Sonicate (FIG. 5)

CD30 expression can be induced via CD28 as well as by IL-4. Therefore in order to prove that the CD30 expression seen in cultures containing M. tuberculosis sonicate is at least partly driven by IL-4, experiments were performed in which either 10 μg/ml anti-human IL-4 antibody or an isotype control antibody was included in the culture medium in some wells and their effects on CD30 expression investigated. The optimal concentration of anti-IL-4 was derived by titration (FIG. 5A inset). CD30 expression was significantly and consistently reduced by anti-IL-4 in MtbS-stimulated lymphocytes of four donors studied in separate experiments (p=0.024 by paired t-test, FIG. 5B). Thus, it was deduced that IL-4 significantly influenced CD30 expression on MtbS-stimulated cells.

FIG. 5. Effect of inhibiting IL-4 on CD30 expression. PBMCs were cultured in the presence of culture medium alone, MvacS or MtbS as previously described. Either anti-IL-4 (10 μg/ml) or isotype control antibody was added to the cultures from Day O, and CD30 expression on lymphogated cells was determined by flow cytometry. The appropriate concentration of anti-IL-4 was determined by titration experiments performed on MtbS-stimulated cells (A inset).

A: One representative experiment out of four performed independently using cells from different donors is shown. Assays were performed in triplicate and presented as means and 2 SD. Statistics by t-test for independent samples.

B: Data from the four independent experiments are shown individually, each point representing the mean of triplicate assays based on cells from one donor. The difference in CD30 expression with and without anti-IL-4 was significant in MtbS-stimulated lymphocytes (p=0.024) but not in MvacS-stimulated lymphocytes (p=0.16). Statisics by paired t-test (n=4).

6) Comparison of Blocking IL-4 and With Anti-IL-4, and Blocking CD28 With CTLA-4/Fc Chimera (FIG. 6)

To further demonstrate that at least part of the CD30 expression is driven by IL-4, the relative effects of inhibiting IL-4 activity and CD28 signalling were next considered by peforming further experiments with CTLA-4/Fc chimeric protein. CTLA-4 binds to CD80 and CD86 with 20-100-fold higher affinity than CD28, thus the chimeric protein acts as a competitive inhibitor of CD28 signalling.

The effect of blocking CD28 signalling was also a significant reduction in CD30 expression (p=0.0004 by paired t-test on data from two independent experiments). However the effect of the anti-IL-4 was of comparable magnitude.

The results are shown in FIG. 6 as flow cytometry dot plots.

FIG. 6. Effects of IL-4 and CD28 on CD30 expression. Anti-IL-4 (10 μg/ml), CTLA-4/Fc (100 μg/ml) or isotype control antibody were added from Day 0 to PBMCs cultured in the presence of MtbS, and CD30 expression determined on Day 7. The flow cytometry dot plots are gated on lymphocytes and numbers in each quadrant indicate the percentage of gated cells in that quadrant. The diagrams illustrate one representative assay of triplicates, in one experiment of three performed independently using cells from different donors.

### SUMMARY

In summary, the present invention relates to vaccines comprising Th2 inducing antigens as described herein.

The invention further relates to adjuvants comprising Th1 inducing entities, and to vaccines comprising same.

The invention also relates to methods for the selection of antigens for use in vaccines as described herein.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

Hernandez-Pando R, Pavon L, Arriaga K, Orozco H, Madrid-Marina V and Rook G A W (1997) Pathogenesis of tuberculosis in mice exposed to low and high doses of an environmental mycobacterial saprophyte. Infect Immun 65:3317-3327.

Hernandez-Pando R and Rook G A W (1994) The role of TNF□ in T cell-mediated inflammation depends on the Th1/Th2 cytokine balance. Immunology 82:591-595.

Lawrence C E, Paterson J C, Higgins L M, MacDonald T T, Kennedy M W and Garside P (1998) IL-4-regulated enteropathy in an intestinal nematode infection. Eur J Immunol 28:2672-2684.

Lindblad E B, Elhay M J, Silva R, Appelberg R and Andersen P (1997) Adjuvant modulation of immune responses to tuberculosis subunit vaccines. Infect Immun 65:623-629.

Munk M E, Kern P and Kaufmann S H (1997) Human CD30+ cells are induced by Mycobacterium tuberculosis and present in tuberculosis lesions. Int Immunol 9:713-20.

Orme I, Flynn J L and Bloom B R (1993) The role of CD8+ T cells in immunity to tuberculosis. Trends Microbiol 1:77-78.

Orme I M, Andersen P and Boom W H (1993) T cell response to Mycobacterium tuberculosis. J Infect Dis 167:1481-1497.

Powrie F, Menon S and Coffman R L (1993) Interleukin-4 and interleukin-10 synergize to inhibit cell-mediated immunity in vivo. Eur J Immunol 23:3043-9.

Seah G T and Rook G A (1999) A sensitive, non-radioactive quantitative method for measuring IL-4 and IL-4delta2 mRNA in unstimulated cells from multiple clinical samples, using nested RT-PCR. J Immunol Methods 228:139-149.

Seah G T, Scott G M and Rook G A (2000) Type 2 Cytokine Gene Activation and Its Relationship to Extent of Disease in Patients with Tuberculosis. J Infect Dis 181:385-389.

Silver R E, Li Q, Boom W H and Ellner J J (1998) Lymphocyte-dependent inhibition of growth of virulent Mycobacterium tuberculosis H37Rv within human monocytes: requirement for CD4+ T cells in purified protein derivative-positive, but not in purified protein derivative-negative subjects. J Immunol 160:2408-17.

Stenger S, Hanson D A, Teitelbaum R, Dewan P, Niazi K R, Froelich C J, Ganz T, S. T-U, Melian A, Bogdan C, Porcelli S A, Bloom B R, Krensky A M and Modlin R L (1998) An antimicrobial activity of cytolytic T cells mediated by granulysin. Science 282:121-125.

van Crevel R, Karyadi E, Preyers F, Leenders M, Kullberg B J, Nelwan R H and van Der Meer J (2000) Increased Production of Interleukin 4 by CD4+ and CD8+ T Cells from Patients with Tuberculosis Is Related to the Presence of Pulmonary Cavities. J Infect Dis 181:1194-1197.

Wynn T A, Cheever A W, Jankovic D, Poindexter R W, Caspar P, Lewis F A and Sher A (1995) An IL-12-based vaccination method for preventing fibrosis induced by schistosome infection. Nature 376:594-596.

1. A pharmaceutical composition comprising a M. tuberculosis antigen capable of inducing Th2, and an adjuvant wherein said adjuvant is capable of inhibiting T-helper cell 2 (Th2) to that antigen.

2. A pharmaceutical composition according to claim 1 wherein said adjuvant is also capable of inducing T-helper cell 1 (Th1) to that antigen.

3. A pharmaceutical composition according to claim 2 wherein said adjuvant comprises IL-12.

4. A pharmaceutical composition according to claim 2 wherein said adjuvant comprises Mycobacterium vaccae, or a part thereof.

5. A pharmaceutical composition according to any of claims 1-4 wherein said composition is a tuberculosis vaccine composition.

6. Use of an adjuvant in a tuberculosis vaccine comprising a Th2 inducing antigen wherein said adjuvant is capable of inhibiting T-helper cell 2 (Th2) to that antigen.

7. Use of an adjuvant according to claim 6 wherein the Th2 inducing antigen is a M. tuberculosis antigen.

8. Use of an adjuvant according to claim 6 or claim 7 wherein said adjuvant is also capable of inducing T-helper cell 1 (Th1) to that antigen.

9. Use of an adjuvant according to claim 8 wherein said adjuvant comprises IL-12.

10. Use of an adjuvant according to claim 8 wherein said adjuvant comprises Mycobacterium vaccae, or a part thereof.

11. A method of identifying an antigen for use in a tuberculosis vaccine, said method comprising
(i) providing a candidate antigen;
(ii) providing a first and a second sample of peripheral blood mononuclear cells (PBMCs);
(iii) contacting said antigen with said first sample of PBMCs;
(iv) monitoring the expression levels of IL-4 in said first and second samples of PBMCs; and
(v) comparing the expression levels of IL-4 of step (iv), wherein antigens inducing raised expression of IL-4 in the first sample of PBMCs as compared to the levels of expression of IL-4 in the second (untreated) sample of PBMCs are identified as useful in said vaccine.

12. A method of identifying an antigen for use in a tuberculosis vaccine, said method comprising
(i) providing a candidate antigen;
(ii) providing a first and a second sample of peripheral blood mononuclear cells (PBMCs);
(iii) contacting said antigen with said first sample of PBMCs;
(iv) monitoring the expression of CD30 in said first and second samples of PBMCs; and
(v) comparing the expression levels of CD30 of step (iv),
wherein antigens inducing raised expression of CD30 in the first sample of PBMCs as compared to the levels of expression of CD30 in the second (untreated) sample of PBMCs are identified as useful in said vaccine.

13. A method according to claim 12 wherein the expression levels of CD30 are IL-4 mediated expression levels.

14. An antigen identified by a method according to any of claims 11 to 13.

15. A vaccine comprising an antigen according to claim 14 and an adjuvant, wherein said adjuvant is capable of inhibiting T-helper cell 2 (Th2) to that antigen.

16. A pharmaceutical composition comprising an antigen as defined in any one of the preceding claims, and an adjuvant, wherein said adjuvant is capable of inhibiting T-helper cell 2 (Th2) to that antigen and optionally further comprising a pharmaceutically acceptable carrier, diluent or excipient.

17. A method for immunising a subject against M. tuberculosis comprising administering to a subject in need of same a vaccine composition according to any preceding claim.

18. A method of inhibiting T-helper cell 2 (Th2) to an antigen in a subject comprising administering to said subject a pharmaceutical composition comprising said antigen according to any preceding claim.

19. A method for the prevention or treatment of M. tuberculosis infection in a subject comprising administering to said subject a vane composition according to any preceding claim.
